(19) United States
(12) Patent Application Publication
(10) Pub. No.: US 2010/0256033 A1
(43) Pub. Date: Oct. 7, 2010

(54) Substrates Having a Cleaning Composition for Improved Glide Over Skin

(76) Inventors: Karen Marie Menard, Neenah, WI (US); Stacy Averic Mundschau, Weyauwega, WI (US); Rhiannon Lynn Thoresen, Appleton, WI (US); Brian Michael Langolf, Fond du Lac, WI (US); Andrew Robert Kischnick, Appleton, WI (US)

Correspondence Address: KIMBERLY-CLARK WORLDWIDE, INC.;Tara Pohlkotte, 2300 Winchester Rd., NEENAH, WI, 54956, US

(21) Appl. No.: 12/646,001

(22) Filed: Dec. 23, 2009

Related U.S. Application Data
(60) Provisional application No. 61/166,923, filed on Apr. 6, 2009.

(51) Int. Cl. A61K 8/02

(52) U.S. Cl. 510/146

(57) ABSTRACT

A cleansing composition including a sarcosine based surfactant is disclosed. Desirably, the cleansing composition contains a sarcosinate surfactant. Additional cleansing surfactants that are nonionic, anionic, cationic, amphoteric, zwitterionic, or combinations thereof, may also be incorporated into the cleansing composition. The cleansing composition may be used in combination with a product, such as a personal care product, to improve aesthetic properties of that product such as glide across the skin. As such, the cleansing product has a coefficient of friction of less than about 0.40. More particularly, cleansing compositions may be incorporated into or onto a substrate, such as a wipe substrate, an absorbent substrate, a fabric or cloth substrate, or a tissue substrate, among others.

This application claims priority from presently copending U.S. Provisional Application No. 61/166,923 entitled “Substrates Having a Cleaning Composition for Improved Glide Over Skin” filed on Apr. 6, 2009, in the names of Karen Marie Menard et al. (Docket No. 64527382US01).

### BACKGROUND

Cleansing substrates have been used in the personal care industry for numerous years, and generally comprise a low surfactant, high water base for cleaning bodily fluids or wiping up menses. In recent years, however, consumers have begun demanding more out of personal care products, including wipes. For example, various wipes have come into the market containing ingredients for soothing skin or containing actives for disinfecting surfaces.

Another example of a desired personal care property is the delivery of perceivable consumer aesthetics such as skin glide. It is desirable for cleansing substrates to deliver good aesthetics to the skin that are perceivable by the consumer. Often, these ingredients are added to reduce the tackiness or residual feel left by surfactant systems or the inherent roughness or abrasive nature of the substrate itself. However, prior attempts to improve the aesthetics of cleansing substrates, such as wet wipes, have proven difficult or limited in terms of manufacturing flexibility and cost.

In particular, many skin benefit agents that may act to improve the feel of the cleansing substrates are hydrophobic, and thus are difficult to effectively incorporate into wet wipe formulations which typically comprise large amounts of water. Prior attempts to overcome the difficulties involved in incorporating hydrophobic skin benefit agents into aqueous wet wipe solutions include, for example, solubilizing, dispersing, or emulsifying oils into a wet wipe solution. These techniques have proven very difficult, however, since stability of oil in a water system is extremely difficult to achieve without separation of the oil. Furthermore, oil on a substrate based product can lead to delamination of certain base sheet structures. The separation issues may be addressed by raising the surfactant concentration in the wet wipe solution, or by incorporating surfactants high in polyethylene glycol (PEG) and/or polypropylene glycol (PPG) to stabilize the oil in the aqueous wet wipe solution over long periods of time. Other emulsifying agents widely known in the art are also used to stabilize the oil within the aqueous solution.

While these approaches may be effective at stabilizing the oil present in the wet wipe solution, there are other drawbacks. In particular, increasing the concentration of surfactant may result in increased irritation to the skin. Even for stabilized emulsion formulations that do not adversely affect the structural and mechanical integrity of the basesheet, producing a concentrated form of the solution is difficult. Thus, the ongoing transportation costs are substantially higher than solutions consisting of water soluble ingredients. To ensure stability of these concentrates, emulsion stabilizing ingredients would need to be added that often substantially increase the solution viscosity, making the solution more difficult to handle in a manufacturing setting. Further, without an effective concentrate of the solution, additional capital equipment to batch the solution and apply it to the wipe within the same site is significantly more complex and costly. Additionally, the skin feel and consumer aesthetics contributed by the oil soluble emollients and emulsifiers improve glide may adversely affect the cleaning performance of the product and impart undesirable residue on the skin such as oiliness.

There are several examples of emulsion systems being applied to a basesheet to cleanse. For example, compositions for personal cleansing have been prepared in the form of oil-in-water emulsions. Within emulsions, silicones are commonly used as both emollients and emulsifiers to provide a desirable skin feel to the product. A specific composition includes an oil-in-water emulsion containing at least one silicone surfactant, at least one oil, and at least one detergent surfactant which may contain no other emulsifier other than the at least one silicone surfactant. The sited advantage of using the silicone surfactant makes it possible to notably improve the makeup-removing capacity of the composition while at the same time leaving no greasy or tacky feeling on the skin. Another cleansing composition is suitable for topical application to human skin, more particularly to an oil-based cleansing composition containing a silicone elastomer gelling agent for removal of make-up from the skin. Another example teaches a method of applying a trisiloxane-containing composition to the surface of a substrate. This class of compounds is widely used to impart improved aesthetics to formulations intended for facial care products.

However, with all these emulsion systems, the overall material costs may be too high and the ultimate ability to concentrate the blend may be too low to provide a commercially viable solution. Further, the overall aesthetic experience for consumers may not be preferred to conventional wipes that provide a light, squeaky clean after feel to the skin with little to no residual feel on the skin.

In addition, water soluble alkyl diols and silicones have also been used and have in part succeeded in overcoming many of these problems for cleansing solutions. For example, modified sorbitan siloxanes may improve the skin glide of wet wipe products. In another example, a disposable substantially dry cleansing article having a lathering surfactant and a C₅₋₁₂ alkyl diol is impregnated into a flexible substrate such as a nonwoven cloth. The alkyl diol primarily is a process aid which may concurrently improve aesthetics and increase lathering. However, the ability of these materials to effectively clean and condition the skin without the presence of a surfactant and glide agent is limited.

Therefore, a highly water soluble ingredient that simultaneously cleans and improves the glide and overall sensation of a cleaning substrate is needed. Further, a low cost and a stable, concentrated, low viscosity solution is needed to provide a commercially feasible cleansing solution.

### SUMMARY

The present disclosure generally relates to a cleansing composition that is applied to a basesheet including a sarcosine based surfactant to improve the glide across the skin. Additional cleansing surfactants that are nonionic, anionic, cationic, amphoteric or zwitterionic may also be incorporated into the cleansing composition.

Desirably, the sarcosine based surfactant is a sarcosinate surfactant. The sarcosine based surfactant may be selected from, but not limited to, ammonium cocoyl sarcosinate, ammonium lauroyl sarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium palmitoyl sarcosinate, sodium sarcosinate, TEA-cocoyl sarcosinate, TEA-lauroyl sarcosinate, TEA-oleoyl sarcosinate, TEA-palm kernel sarcosinate, and combinations thereof. Desirably, the sarcosine based surfactant is sodium lauroyl sarcosinate.

Typically, the cleansing composition contains from about 0.01% (by weight of the cleansing composition) to about 95% (by weight of the cleansing composition) of the sarcosine based surfactant. Desirably, the cleansing composition contains from about 0.01% (by weight of the cleansing composition) to about 10% (by weight of the cleansing composition) of the sarcosine based surfactant. Even more desirably, the cleansing composition contains from about 0.01% (by weight of the cleansing composition) to about 5% (by weight of the cleansing composition) of the sarcosine based surfactant.

The cleaning composition further comprises an additional surfactant selected from surfactants including nonionic, anionic, cationic, amphoteric, zwitterionic, or combinations thereof. Typically, the cleansing composition can contain additional surfactants in an amount of from about 0.01% (by weight cleansing composition) to about 90% (by weight cleansing composition). More suitably, the additional surfactant is present in the cleansing composition in an amount of from about 0.1% (by weight cleansing composition) to about 15% (by weight cleansing composition).

The cleansing composition may further contain a skin benefit agent selected from the group consisting of a quaternary ammonium material, a particulate, a rheology modifier, a moisturizer, a film former, a slip modifier, a surface modifier, a skin protectant, a sunscreen, and combinations thereof.

### DETAILED DESCRIPTION

The present disclosure generally relates to a cleansing composition including a sarcosine based surfactant. Desirably, the sarcosine based surfactant is a sarcosinate surfactant. Additional cleansing surfactants that are nonionic, anionic, cationic, amphoteric, zwitterionic, or combinations thereof, may also be incorporated into the cleansing composition. The cleansing composition of the present disclosure may be used in combination with a product, such as a personal care product to improve aesthetic properties of that product such as glide across the skin. More particularly, the cleansing composition may be incorporated into or onto a substrate, such as a wipe substrate, an absorbent substrate, a fabric or cloth substrate, or a tissue substrate, among others. For example, the compositions may be incorporated into personal care products, such as wipes, mitts, absorbent articles, bath tissues, cloths, and the like. More particularly, the cleansing composition may be incorporated into wipes such as wet wipes, dry wipes, hand wipes, face wipes, cosmetic wipes, and the like, or absorbent articles, such as diapers, training pants, adult incontinence products, feminine hygiene products, and the like. Desirably, the cleansing composition is a liquid composition that may be used in combination with a wipe substrate to form a wet wipe, or may be a wetting composition for use in combination with a dispersible wet wipe.

Sarcosine based surfactants for use in the cleansing composition are mild anionic surfactants that can be used in a wide range of personal care applications. Desirably, the sarcosine based surfactant is a sarcosinate surfactant. Several appropriate applications of the sarcosinate salts include, but are not limited to, shampoos, mild facial cleansers, body washes and foaming bath products, liquid soaps, antibacterial hand washes, shaving preparations and liquid makeup. Sarcosinates provide enhanced lathering properties of cleansers, offer mild detergency while exhibiting synergy with other detergents, are compatible with various cationics, including conditioning agents and are reported to be substantive to skin and hair. Non-limiting examples of suitable sarcosine based surfactants include ammonium cocoyl sarcosinate, ammonium lauroyl sarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium palmitoyl sarcosinate, sodium sarcosinate, TEA-cocoyl sarcosinate, TEA-lauroyl sarcosinate, TEA-oleoyl sarcosinate, TEA-palm kernel sarcosinate, and combinations thereof. Desirably, the sarcosine based surfactant may be sodium lauroyl sarcosinate.

Typically, the cleansing composition contains from about 0.01% (by weight of the cleansing composition) to about 95% (by weight of the cleansing composition) of the sarcosine based surfactant. Desirably, the cleansing composition contains from about 0.01% (by weight of the cleansing composition) to about 10% (by weight of the cleansing composition) of the sarcosine based surfactant. Even more desirably, the cleansing composition contains from about 0.01% (by weight of the cleansing composition) to about 5% (by weight of the cleansing composition) of the sarcosine based surfactant. In an exemplary cleansing composition, the additional surfactant is alkyl polyglucoside. Desirably, the additional surfactant is sodium lauryl glucose carboxylate.

As described above, the cleansing composition may also contain a nonionic surfactant as an additional cleansing surfactant. Nonionic surfactants typically have a hydrophobic base, such as a long chain alkyl group or an alkylated aryl group, and a hydrophilic chain comprising a certain number (e.g., 1 to about 30) of ethoxy and/or propoxy moieties. Examples of some classes of nonionic surfactants that can be used with the cleansing composition include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, and mixtures thereof.

Examples of suitable nonionic surfactants include, but are not limited to, methyl gluceth-10, PEG-20 methyl glucose distearate, PEG-20 methyl glucose sesquistearate, C₁₁₋₁₅ pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₈₋₂₂) alcohol, including 3 to 20 ethylene oxide moieties, polyoxyethylene-20 isohexadecyl ether, polyoxyethylene-23 glycerol laurate, PEG 80 sorbitan laurate, polyoxy-ethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxy-ethylene-6 tridecyl ether, laureth-2, laureth-3, laureth-4, PEG-3 castor oil, PEG 600 dioleate, PEG 400 dioleate, and combinations thereof.

Specific examples of such commercially available nonionic surfactants of the foregoing type are C₁₁₋₁₅ secondary alkanols condensed with either 9 moles of ethylene oxide (Tergitol 15-S-9) or 12 moles of ethylene oxide (Tergitol 15-S-12) commercially available from Union Carbide Corporation, Danbury, Conn. Other suitable nonionic surfactants include the polyethylene oxide condensates of one mole of alkyl phenol containing from about 8 to 18 carbon atoms in a straight or branched chain alkyl group with about 5 to 30 moles of ethylene oxide. Specific examples of alkyl phenol ethoxylates include nonyl condensed with about 9.5 moles of ethylene oxide per mole of nonyl phenol, dinonyl phenol condensed with about 12 moles of ethylene oxide per mole of phenol, dinonyl phenol condensed with about 15 moles of ethylene oxide per mole of phenol and diisoctylphenol condensed with about 15 moles of ethylene oxide per mole of phenol. Commercially available nonionic surfactants of this type include Igepal CO-630 (a nonyl phenol ethoxylate) commericially available from ISP Corporation, Wayne, N.J. Suitable non-ionic ethoxylated octyl and nonyl phenols include those having from about 7 to about 13 ethoxy units. Such compounds are commercially available under the trade name Triton X commercially available from Union Carbide, Danbury, Conn.). Alkyl polyglycosides may also be used as a nonionic surfactant in the present cleansing compositions. Suitable alkyl polyglycosides are known nonionic surfactants that are alkaline and electrolyte stable. Alkyl mono and polyglycosides are prepared generally by reacting a monosaccharide, or a compound hydrolyzable to a monosaccharide with an alcohol such as a fatty alcohol in an acid medium. One example of such alkyl polyglycosides is APG™ 325 CS GLYCOSIDE, which is described as being a 50% C₉₋₁₁ alkyl polyglycoside, also commonly referred to as D-glucopyranoside. Another example of an alkyl polyglycoside surfactant is GLUCOPON™ 625 CS, which is described as being a 50% C₁₀₋₁₆ alkyl polyglycoside, also commonly referred to as a D-glucopyranoside. Both APG™ 325 CS GLYCOSIDE and GLUCOPON™ 625 CS are commercially available from COGNIS Corporation, Ambler, Pa.

As described above, anionic surfactants may be utilized as an additional cleansing surfactant within the cleansing composition. Suitable anionic surfactants include, but are not limited to, alkyl sulfates, alkyl ether sulfates, alkyl ether sulfonates, sulfate esters of an alkylphenoxy polyoxyethylene ethanol, alpha-olefin sulfonates, beta-alkoxy alkane sulfonates, alkylauryl sulfonates, alkyl monoglyceride sulfates, alkyl monoglyceride sulfonates, alkyl carbonates, alkyl ether carboxylates, fatty acids, sulfosuccinates, phosphates, taurates, fatty taurides, fatty acid amide polyoxyethylene sulfates, isethionates, or combinations thereof.

Examples of some suitable anionic surfactants include, but are not limited to, C₈₋₁₈ alkyl sulfates, C₈₋₁₈ fatty acid salts, C₈₋₁₈ alkyl ether sulfates having one or two moles of ethoxylation, C₈₋₁₈ alkamine oxides, C₈₋₁₈ sulfoacetates, C₈₋₁₈ sulfosuccinates, C₈₋₁₈ alkyl diphenyl oxide disulfonates, C₈₋₁₈ alkyl carbonates, C₈₋₁₈ alpha-olefin sulfonates, methyl ester sulfonates, and blends thereof. The C₈₋₁₈ alkyl group can be straight chain (e.g., lauryl) or branched (e.g., 2-ethylhexyl). The cation of the anionic surfactant can be an alkali metal (e.g., sodium or potassium), ammonium, C₁₋₄ alkylammonium (e.g., mono-, di-, tri-), or C₁₋₃ alkanolammonium (e.g., mono-, di-, tri-). Specific examples of such anionic surfactants include, but are not limited to, lauryl sulfates, octyl sulfates, 2-ethylhexyl sulfates, lauramine oxide, decyl sulfates, tridecyl sulfates, cocoates, lauryl sulfosuccinates, linear C₁₀ diphenyl oxide disulfonates, lauryl sulfosuccinates, lauryl ether sulfates (1 and 2 moles ethylene oxide), myristyl sulfates, oleates, stearates, tallates, phosphates ricinoleates, cetyl sulfates, and similar surfactants. Desirably, the additional surfactant is sodium lauryl glucose carboxylate.

Cationic surfactants, such as cetylpyridinium chloride and methylbenzethonium chloride, may also be utilized provided they are found to be compatible with the sarcosine based surfactant.

The cleansing composition may also contain other types of surfactants. For instance, in some embodiments, amphoteric surfactants, such as zwitterionic surfactants, may also be used. For instance, one class of amphoteric surfactants that may be used in the present disclosure are derivatives of secondary and tertiary amines having aliphatic radicals that are straight chain or branched, wherein one of the aliphatic substituents contains from about 8 to 18 carbon atoms and at least one of the aliphatic substituents contains an anionic water-solubilizing group, such as a carboxy, sulfonate, or sulfate group. Some examples of amphoteric surfactants include, but are not limited to, sodium 3-(dodecylamino)propionate, sodium 3-(dodecylamino)-propane-1-sulfonate, sodium 2-(dodecylamino)ethyl sulfate, sodium 2-(dimethylamino)octadecanoate, disodium 3-(N-carboxymethyl-dodecylamino)propane-1-sulfonate, disodium octadecyliminodiacetate, sodium 1-carboxymethyl-2-undecylimidazole, and sodium N,N-bis(2-hydroxyethyl)-2-sulfato-3-dodecoxypropylamine. Additional classes of suitable amphoteric surfactants include phosphobetaines and the phosphitaines. For instance, some examples of such amphoteric surfactants include, but are not limited to, sodium coconut N-methyl taurate, sodium oleyl N-methyl taurate, sodium tall oil acid N-methyl taurate, sodium palmitoyl N-methyl taurate, cocodimethylcarboxymethylbetaine, lauryldimethylcarboxymethylbetaine, lauryldimethylcarboxyethylbetaine, cetyldimethylcarboxymethylbetaine, lauryl-bis-(2-hydroxyethyl)carboxymethylbetaine, oleyldimethylgammacarboxypropylbetaine, lauryl-bis-(2-hydroxypropyl)-carboxyethylbetaine, cocoamidodimethylpropylsultaine, stearylamidodimethylpropylsultaine, laurylamido-bis-(2-hydroxyethyl)propylsultaine, di-sodium oleamide PEG-2 sulfosuccinate, TEA oleamido PEG-2 sulfosuccinate, disodium oleamide MEA sulfosuccinate, disodium oleamide MIPA sulfosuccinate, disodium ricinoleamide MEA sulfosuccinate, disodium undecylenamide MEA sulfosuccinate, disodium lauryl sulfosuccinate, disodium wheat germamido MEA sulfosuccinate, disodium wheat germamido PEG-2 sulfosuccinate, disodium isostearamideo MEA sulfosuccinate, cocoamphoglycinate, cocoamphocarboxyglycinate, lauroamphoglycinate, lauroamphocarboxyglycinate, capryloamphocarboxyglycinate, cocoamphopropionate, cocoamphocarboxypropionate, lauroamphocarboxypropionate, capryloamphocarboxypropionate, dihydroxyethyl tallow glycinate, cocoamido disodium 3-hydroxypropyl phosphobetaine, lauric myristic amido disodium 3-hydroxypropyl phosphobetaine, lauric myristic amido glyceryl phosphobetaine, lauric myristic amido carboxy disodium 3-hydroxypropyl phosphobetaine, cocoamido propyl monosodium phosphitaine, cocamidopropyl betaine, lauric myristic amido propyl monosodium phosphitaine, and mixtures thereof.

Other useful zwitterionic surfactants include compositions based on amine oxides. One general class of useful amine oxides include alkyl di(lower alkyl) amine oxides in which the alkyl group has about 10 to 20, and preferably 12 to 16 carbon atoms, and can be straight or branched chain, saturated or unsaturated. The lower alkyl groups include between 1 and 7 carbon atoms. Examples include lauryl, dimethyl amine oxide, myristyl dimethyl amine oxide, and those in which the alkyl group is a mixture of different amine oxide, dimethyl cocoamine oxide, dimethyl (hydrogenated tallow) amine oxide, and myristyl/palmityl dimethyl amine oxide. Another class of useful amine oxides include alkyl di(hydroxy lower alkyl) amine oxides in which the alkyl group has about 10 to 20, and particularly 12 to 16 carbon atoms, and can be straight or branched chain, saturated or unsaturated. Examples are bis(2-hydroxyethyl) cocoamine oxide, bis(2-hydroxyethyl) tallow amine oxide, and bis(2-hydroxyethyl) stearylamine oxide. Moreover, still other useful amine oxides include those characterized as alkylamidopropyl di(lower alkyl) amine oxides, in which the alkyl group has about 10 to 20 carbon atoms, and can be straight or branched chain, saturated or unsaturated. Examples are cocoamidopropyl dimethyl amine oxide and tallowamidopropyl dimethyl amine oxide. Additional useful amine oxides include alkylmorpholine oxides in which the alkyl group has about 10 to 20 carbon atoms, and can be straight or branched chain, saturated or unsaturated. Further examples of amine oxides include those that commercially available under the trade name AMMONYX from Stepan Company, Chicago, Ill.

The amount of additional surfactants contained in the cleansing composition can vary greatly depending upon various factors. Typically, the cleansing composition can contain additional surfactants in an amount of from about 0.01% (by weight cleansing composition) to about 90% (by weight cleansing composition). More suitably, the additional surfactant is present in the cleansing composition in an amount of from about 0.1% (by weight cleansing composition) to about 15% (by weight cleansing composition).

In one embodiment, the cleansing compositions may comprise water. The cleansing compositions can suitably comprise water in an amount of from about 0.1% (by weight of the composition) to about 99.5% (by weight of the cleansing composition) and more preferably from about 60% (by weight of the cleansing composition) to about 99.5% (by weight of the cleansing composition). For instance, where the composition is used in connection with a wet wipe, the composition can suitably comprise water in an amount of from about 75% (by weight of the cleansing composition) to about 99.5% (by weight of the cleansing composition).

The cleansing composition may further contain additional agents that impart a beneficial effect on skin and/or further act to improve the aesthetic feel of the compositions and wipes described herein. Examples of suitable skin benefit agents include emollients, sterols or sterol derivatives, natural and synthetic fats or oils, rheology modifiers, polyols, surfactants, alcohols, esters, silicones, clays, starch, cellulose, particulates, moisturizers, film formers, slip modifiers, surface modifiers, skin protectants, humectants, sunscreens, and combinations thereof.

The cleansing composition may further optionally include one or more emollients, which typically act to soften, soothe, and otherwise lubricate and/or moisturize the skin. Suitable emollients that can be incorporated into the cleansing composition include oils such as petrolatum based oils, petrolatum, vegetable based oils, mineral oils, natural or synthetic oils, alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty esters, glycerol esters and derivatives, propylene glycol esters and derivatives, alkoxylated carboxylic acids, alkoxylated alcohols, fatty alcohols, and combinations thereof. Suitable esters could include, but not be limited to, cetyl palmitate, stearyl palmitate, cetyl stearate, isopropyl laurate, isopropyl myristate, isopropyl palmitate, and combinations thereof. The fatty alcohols could include, but not be limited to, octyldodecanol, lauryl, myristyl, cetyl, stearyl, behenyl alcohol, and combinations thereof. Ethers such as eucalyptol, cetearyl glucoside, dimethyl isosorbic polyglyceryl-3 cetyl ether, polyglyceryl-3 decyltetradecanol, propylene glycol myristyl ether, and combinations thereof can also suitably be used as emollients. The cleansing composition may include one or more emollients in an amount of from about 0.01% (by weight of the composition) to about 10% (by weight of the composition), more desirably from about 0.05% (by weight of the composition) to about 5% (by weight of the composition), and even more desirably from about 0.1% (by weight of the composition) to about 1% (by weight of the composition).

Sterol and sterol derivatives which are suitable for use in the cleansing composition include, but are not limited to cholesterol, sitosterol, stigmasterol, ergosterol, C₁₀₋₃₀ cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyidecanoate, ergocalciferol, tall oil sterol, soy sterol acetate, lanasterol, soy sterol, avocado sterols, fatty alcohols, and combinations thereof. The cleaning composition can include sterols, sterol derivatives or mixtures of both sterols and sterol derivatives in an amount of from about 0.01% (by weight of the composition) to about 10% (by weight of the composition), more desirably from about 0.05% (by weight of the composition) to about 5% (by weight of the composition), and even more desirably from about 0.1% (by weight of the composition) to about 1% (by weight of the composition).

The compositions of the disclosure can also include natural fats and oils. As used herein, the term “natural fat or oil” is intended to include fats, oils, essential oils, essential fatty acids, non-essential fatty acids, phospholipids, and combinations thereof. These natural fats and oils can provide a source of essential and non-essential fatty acids to those found in the skin's natural barrier. Suitable natural fats or oils can include, but are not limited to, citrus oil, olive oil, avocado oil, apricot oil, babassu oil, borage oil, camellia oil, canola oil, castor oil, coconut oil, corn oil, cottonseed oil, emu oil, evening primrose oil, hydrogenated cottonseed oil, hydrogenated palm kernel oil, jojoba oil, maleated soybean oil, meadowfoam oil, palm kernel oil, peanut oil, rapeseed oil, grapeseed oil, safflower oil, sphingolipids, sweet almond oil, tall oil, lauric acid, palmitic acid, stearic acid, linoleic acid, stearyl alcohol, lauryl alcohol, myristyl alcohol, behenyl alcohol, rose hip oil, calendula oil, chamomile oil, eucalyptus oil, juniper oil, sandlewood oil, tea tree oil, sunflower oil, soybean oil, and combinations thereof. The cleansing composition may include fats and oils in an amount of from about 0.01% (by weight of the composition) to about 10% (by weight of the composition), more desirably from about 0.01% (by weight of the composition) to about 5% (by weight of the composition), and even more desirably from about 0.1% (by weight of the composition) to about 1% (by weight of the composition).

The cleansing composition may further comprise other additional additives to improve a functional or physical property of the composition. For example, the cleansing composition may include one or more non-aqueous solvents. Although not required, non-aqueous solvents can sometimes aid in dissolving certain components (e.g., preservatives, anti-microbial agent, etc.). Examples of some suitable non-aqueous solvents include, but are not limited to, glycerin; glycols, such as propylene glycol, butylene glycol, triethylene glycol, hexylene glycol, polyethylene glycols, ethoxydiglycol, and dipropyleneglycol; alcohols, such as ethanol, n-propanol, and isopropanol; triglycerides; ethyl acetate; acetone; triacetin; and combinations thereof. Solvent combinations include a glycol, particularly hexylene and/or propylene glycol, and one or more lower alcohols, particularly isopropanol, n-propanol, and/or ethanol.

The cleansing composition may optionally further comprise humectants. Examples of suitable humectants include glycerin, glycerin derivatives, sodium hyaluronate, betaine, amino acids, glycosaminoglycans, honey, sorbitol, glycols, polyols, sugars, hydrogenated starch hydrolysates, salts of PCA, lactic acid, lactates, and urea. A particularly preferred humectant is glycerin. The cleansing composition may suitably include one or more humectant in an amount of from about 0.05% (by weight of the cleansing composition) to about 25% (by weight of the cleansing composition).

The cleansing composition can also include various preservatives to increase the shelf life of the composition. Some suitable preservatives that can be used in the present disclosure include, but are not limited to, Kathon CG, which is a mixture of methylchloroisothiazolinone and methylisothiazolinone, and Nelone 950, which is methylisothiazolinone, both commercially available from Rohm & Haas, Philadelphia, Pa.; Mackstat H 66, commercially available from McIntyre Group, Chicago, Ill.; DMDM hydantoin (e.g., Glydant Plus commercially available from Lonza, Inc., Fair Lawn, N.J.); tetrasodium EDTA; iodopropynyl butylcarbamate; benzoic esters (parabens), such as methylparaben, propylparaben, butylparaben, ethylparaben, isopropyl paraben, isobutylparaben, benzylparaben, sodium methylparaben, and sodium propylparaben; 2-bromo-2-nitropropane-1,3-diol; benzoic acid and its salts; sorbic acid and its salts; amidazolidinyl urea; diazolidinyl urea; and the like. Other suitable preservatives include Germall 115 (amidazolidinyl urea), Germall II (diazolidinyl urea), and Germall Plus (diazolidinyl urea and iodopropynyl butylcarbonate), all commercially available from Sutton Labs, Chatham, N.J., and phenoxyethanol, low molecular glycols such as hexylene and caprylyl glycol, tropolone, and blends thereof. When utilized, the amount of the preservative in the cleansing composition can generally vary depending on the relative amounts of the other components present within the composition. For example, in some embodiments, the preservative is present in the cleansing composition in an amount between about 0.001% (by weight of the cleansing composition) to about 5% (by weight of the cleansing composition), desirably between about 0.001% (by weight of the cleansing composition) to about 1% (by weight of the cleansing composition), and more desirably, between about 0.1% (by weight of the cleansing composition) to about 0.5% (by weight of the cleansing composition).

Moreover, some examples of acidic pH modifiers that may be used in the present disclosure include, but are not limited to, mineral acids; and carboxylic acids; and polymeric acids. Specific examples of suitable mineral acids are hydrochloric acid, nitric acid, phosphoric acid, and sulfuric acid. Specific examples of suitable carboxylic acids are citric acid, glycolic acid, lactic acid, maleic acid, malic acid, succinic acid, glutaric acid, benzoic acid, malonic acid, salicylic acid, gluconic acid, and mixtures thereof. Specific examples of suitable polymeric acids include straight chain poly(acrylic) acid and its copolymers (e.g., maleic-acrylic, sulfonic-acrylic, and styrene-acrylic copolymers), cross linked polyacrylic acids having a molecular weight of less than about 250,000, poly(methacrylic) acid, and naturally occurring polymeric acids such as carageenic acid and alginic acid.

The cleansing composition may additionally include one or more sequestrants or chelants or chelating agents. A sequestrant is a substance whose molecules can form one or more bonds with a metal ion. In particular, water often contains metal ions, such as calcium ions, that might react with anionic components (e.g., surfactants, acids, etc.) present within the cleansing composition. For example, in one embodiment, a surfactant that remains substantially unreacted with metal ions can better function as a cleansing agent. Some examples of sequestrants that may be used in the cleansing composition of the present disclosure include, but are not limited to, ethylenediamines, ethylenediaminetetraacetic acids (EDTA) acid and/or salts thereof, citric acids and/or salts thereof, glucuronic acids and/or salts thereof, polyphosphates, organophosphates, dimercaprols, and the like. Typically, when one or more sequestrants are used in the cleansing compositions, the cleansing composition includes the sequestrants in an amount of from about 0.01% (by weight of the cleansing composition) to about 2% (by weight of the cleansing composition). More suitably, the cleansing composition includes from about 0.05% (by weight of the cleansing composition) to about 1% (by weight of the cleansing composition) sequestrant. More suitably, the cleansing composition includes from about 0.05% (by weight of the cleansing composition) to about 1% (by weight of the cleansing composition) sequestrant.

Additionally, rheology modifiers may be used to alter the consistency, flow properties and even skin aesthetics of the formulation. Ideally, these materials are readily incorporated into a concentrated form of the invention so as to minimize manufacturing complexity and equipment handling requirements. Additionally, many rehology modifiers may also function to suspend oils mentioned within this specification in the solution. Generally, the optimal rheology modifier will modify the target properties of the solution without causing the viscosity to increase beyond 5,000 centipoise. Suitable, non-limiting examples of rheology modifiers include gum arabic, carboxymethylcellulose, carboxymethylpropylcellulose, carregeanan, chitosan, microcrystalline cellulose, cellulose gum, magnesium aluminum silicate, bentonite, hectorite and other appropriate materials. Preferred use levels of these materials is 0.01% to 30%, more preferred 0.01% to 5% and even more preferred 0.01% to 0.5%.

In order to better enhance the cleansing composition, other optional ingredients can also be used. For instance, some classes of ingredients that can be used include, but are not limited to: anti-microbial agents; antioxidants (product integrity); anti-reddening agents, such as aloe extract; astringents—cosmetic (induce a tightening or tingling sensation on skin); astringents—drug (a drug product which checks oozing, discharge, or bleeding when applied to skin or mucous membrane and works by coagulating protein); biological additives (enhance the performance or consumer appeal of the product); deodorants (reduce or eliminate unpleasant odor and protect against the formation of malodor on body surfaces); external analgesics (a topically applied drug that has a topical analgesic, anesthetic, or antipruritic effect by depressing cutaneous sensory receptors, of that has a topical counterirritant effect by stimulating cutaneous sensory receptors); film formers (to hold active ingredients on the skin by producing a continuous film on skin upon drying); fragrances (consumer appeal); hydrotropes (helps dissolve some anti-microbial agents); opacifiers (reduce the clarity or transparent appearance of the product); skin conditioning agents; skin exfoliating agents (ingredients that increase the rate of skin cell turnover such as alpha hydroxy acids and beta hydroxyacids); skin protectants (a drug product which protects injured or exposed skin or mucous membrane surface from harmful or annoying stimuli); sunscreens and thickeners (to increase the viscosity of the formulation). Additional ingredients may be added to create consumer appeal. Non-limiting examples of such ingredients include vitamins such as tocopherol, tocopherol acetate and the retinoids, particularly retinyl palmitate, and plant extracts, such as those derived from shea butter, chamomile, lavender, aloe vera, cucumber, green tea and oatmeal.

The cleansing composition may desirably be incorporated into the wipe in an add-on amount of from about 10% (by weight of the treated substrate) to about 600% (by weight of the treated substrate), more desirably from about 50% (by weight of the treated substrate) to about 500% (by weight of the treated substrate), even more desirably from about 100% (by weight of the treated substrate) to about 400% (by weight of the treated substrate), and especially more desirably from about 200% (by weight of the treated substrate) to 350% (by weight of the treated substrate).

The desired cleansing composition add-on amounts may vary depending on the composition of the wipe substrate. Typically, however, for coform basesheets, the composition add-on amount will be from about 250% (by weight of the treated substrate) to about 350% (by weight of the treated substrate), and more typically about 330% (by weight of the treated substrate). For air-laid basesheets, the composition add-on amount will typically be from about 200% (by weight of the treated substrate) to about 300% (by weight of the treated substrate), and more typically will be about 235% (by weight of the treated substrate).

Further, the cleansing composition is able to be concentrated to a minimum of 10 times the level of use while remaining stable during transportation and storage prior to dilution. More preferred, the cleansing composition is concentrated to 20 times the original composition and even more preferred 50 times the original composition.

As noted above, the cleansing composition including the sarcosine based surfactant may be incorporated into a substrate to improve the perceivable aesthetics of these substrates. One example of a perceivable aesthetic benefit achieved by incorporating the sarcosine based surfactant into a substrate is improved glide of the substrate across the skin as compared to traditional personal care products. Improved glide across the skin provides less friction as the wipe glides across the skin. Less friction as the wipe glides across the skin may be less irritating to sensitive or damaged skin.

In particular, static coefficient of friction values can be used as an indication of shear forces that occur between the skin and materials that may contact the skin, such as a wipe. Lower static coefficient of friction values indicate lower shear forces between the skin and the material, and an increased feeling of gentleness of the wipe and improved ability of the wipe to glide across the skin.

Typically, the coefficient of friction value for a wipe incorporating a sarcosine based surfactant of the present disclosure will be less than about 0.4, more desirably from about 0.3 to about 0.37. Coefficient of friction values may be measured as described in the test method section.

In one particular aspect, the substrate may be wet wipes. Generally, the wipes of the present disclosure including the sarcosine based surfactant can be wet wipes or dry wipes. As used herein, the term “wet wipe” means a wipe that includes greater than about 70% (by weight of the substrate) moisture content. As used herein, the term “dry wipe” means a wipe that includes less than about 10% (by weight of the substrate) moisture content. Specifically, suitable wipes for use in the present disclosure can include wet wipes, dry wipes, hand wipes, face wipes, cosmetic wipes, household wipes, industrial wipes, and the like. Particularly preferred wipes are wet wipes, and other wipe-types that include a solution.

Materials suitable for the substrate of the wipes are well know to those skilled in the art, and are typically made from a fibrous sheet material which may be either woven or nonwoven. For example, suitable materials for use in the wipes may include nonwoven fibrous sheet materials, which include meltblown, coform, air-laid, bonded-carded web materials, hydroentangled materials, and combinations thereof. Such materials can be comprised of synthetic or natural fibers, or combinations thereof. Typically, the wipes of the present disclosure define a basis weight of from about 25 grams per square meter to about 120 grams per square meter and desirably from about 40 grams per square meter to about 90 grams per square meter.

The substrate may comprise a coform basesheet of polymer fibers and absorbent fibers having a basis weight of from about 25 to about 150 grams per square meter and desirably about 60 grams per square meter. Such coform basesheets are manufactured generally as described in U.S. Pat. No. 4,100,324, issued to Anderson, et al.; U.S. Pat. No. 5,284,703, issued to Everhart, et al.; and U.S. Pat. No. 5,350,624, issued to Georger, et al., which are incorporated by reference to the extent to which they are consistent herewith. Typically, such coform basesheets comprise a gas-formed matrix of thermoplastic polymeric meltblown fibers and cellulosic fibers. Various suitable materials may be used to provide the polymeric meltblown fibers, such as, for example, polypropylene microfibers. Alternatively, the polymeric meltblown fibers may be elastomeric polymer fibers, such as those provided by a polymer resin. For instance, Vistamaxx® elastic olefin copolymer resin designated PLTD-1810, available from ExxonMobil Corporation, Houston, Tex.; or KRATON G-2755, commercially available from Kraton Polymers, Houston, Tex. may be used to provide stretchable polymeric meltblown fibers for the coform basesheets. Other suitable polymeric materials, or combinations thereof, may alternatively be utilized as known in the art.

The coform basesheet additionally may comprise various absorbent cellulosic fibers, such as, for example, wood pulp fibers. Suitable absorbents include, but are not limited to, fibrous organic materials, such as woody or non-woody pulp such as cotton, unbleached chlorine free pulp, rayon, recycled paper, wood pulp fluff, cellulose and/or cellulosic staple fibers, and also include inorganic absorbent materials such as superabsorbent materials and/or treated polymeric staple fibers. Suitable cellulose materials are, for example, absorbent cellulosic materials, such as, wood pulp fibers. Suitable commercially available cellulosic fibers for use in the coform basesheets can include, for example, NF 405, which is a chemically treated bleached southern softwood Kraft pulp, commercially available from Weyerhaeuser Company, Washington, D.C.; NB 416, which is a bleached southern softwood Kraft pulp, commercially available from Weyerhaeuser Company; CR-0056, which is a fully debonded softwood pulp, commercially available from Bowater, Inc., Greenville, S.C.; Golden Isles 4822 debonded softwood pulp, available from Koch Cellulose, Brunswick, Ga.; and SULPHATATE HJ, which is a chemically modified hardwood pulp commercially available from Rayonier, Inc., Jesup, Ga.

Other suitable basesheet materials for the substrate include meltblown webs, spunbonded webs, bonded carded webs, textured basesheets such as single wire texture, wet-laid webs, airlaid webs, hydraulically entangled webs, and combinations thereof. Spunlacing is a process of entangling a web of loose fibers on a porous belt or moving perforated or patterned screen to form a sheet structure by subjecting the fibers to multiple rows of fine high-pressure jets of water so that the fibers knot about one another. As a result, nonwoven fabrics made by this method have specific properties, such as soft handle and drapability. An airlaid web is a well known process by which a fibrous nonwoven layer can be formed. In the airlaying process, bundles of small fibers are separated and entrained in an air supply and then deposited onto a forming screen, usually with the assistance of a vacuum supply. The randomly deposited fibers are then bonded to one another using, for example, hot air or a spray adhesive. In another embodiment, the wipe substrate may be an airlaid nonwoven fabric. The basis weights for airlaid nonwoven fabrics may range from about 20 to about 200 grams per square meter with staple fibers having a denier of about 0.5 to 10 and a length of about 6 to 15 millimeters. Wet wipes may generally have a fiber density of about 0.025 g/cc to about 0.2 g/cc. Wet wipes may generally have a basis weight of about 20 grams per square meter to about 150 grams per square meter. More desirably the basis weight may be from about 30 to about 90 grams per square meter. Even more desirably the basis weight may be from about 50 grams per square meter to about 75 grams per square meter. Processes for producing airlaid nonwoven basesheets are described in, for example, published U.S. Patent Application Publication 2006/0008621, herein incorporated by reference.

If desired, the nonwoven web may also be bonded using techniques well known in the art to improve the durability, strength, hand, aesthetics, texture, and/or other properties of the fabric. For instance, the nonwoven web may be thermally (e.g., pattern bonded), ultrasonically, adhesively and/or mechanically (e.g., through-air dried) bonded. For instance, various pattern bonding techniques are described in U.S. Pat. No. 3,855,046 issued to Hansen; U.S. Pat. No. 5,620,779 issued to Levy, et al; and U.S. Pat. No. 5,962,112 issued to Haynes et al., which are all incorporated herein by reference to the extent they are consistent herewith. The nonwoven web may be bonded by continuous seams or patterns. As additional examples, the nonwoven web may be bonded along the periphery of the sheet or simply across the width or cross direction of the web adjacent to the edges. Other bonding techniques, such as a combination of thermal bonding and latex impregnation, may also be used. Alternatively and/or additionally, a resin, latex or adhesive may be applied to the nonwoven fabric by, for example, spraying or printing, and dried to provide the desired bonding.

The relative percentages of the absorbent material can vary over a wide range depending upon the desired characteristics of the cleansing product. For example, the coform basesheet may comprise from about 10 weight percent to about 90 weight percent, desirably from about 20 weight percent to about 60 weight percent, and more desirably from about 25 weight percent to about 35 weight percent of the polymeric meltblown fibers based on the dry weight of the coform basesheet being used to provide the wipes. Typically, the wipes of the present disclosure define a basis weight of from about 25 grams per square meter to about 120 grams per square meter and desirably from about 40 grams per square meter to about 90 grams per square meter.

Additionally, the wet wipe may be water-dispersible when introduced into a waste water stream following use. U.S. Pat. No. 6,960,371 issued to Bunyard et al., which describes the use of ion-sensitive or triggerable, water-dispersible or water-soluble cationic polymers and polymer formulations, is incorporated herein by reference to the extent it is consistent herewith. Despite their anionic character, salts of sarcosine based surfactants are known to be compatible with cationic compounds.

In another suitable embodiment, the substrate is used in the removal of make-up. Suitable facial cleansers are particularly difficult to formulate as the composition must effectively remove compositions such as foundations, blushes, lipstick and eyeliner that are formulated to remain on the skin for extended periods of time and in the presence of water. However, due to the relatively high proportion of the surface area occupied by muscosal tissue, the cleansing composition needs to be very mild. This is important both from the standpoint of the irritation potential of the solution as well as the overall softness and ease with which the product glides across the skin during cleansing. Another key limitation is that facial products must be aesthetically pleasing to consumer during and after use. The optimal cleansing substrate will leave the skin feeling clean, light and conditioned without feeling overly dried and stripped of natural oils and lipids. Use of sarcosine based surfactants to improve the skin glide of make-up removal wipes combined with its well known mildness and cleansing ability make the sarcosine based surfactants suitable for the removal of make-up.

In another embodiment, the cleansing substrate is a wash mitt. Use of disposable wash mitts to aid in child cleansing is well known in the art. In the past reusable washcloths and sponges have been made in various shapes, such as puppets, with child appealing graphics and fragrances or aromas in order to make the use of these products fun and enjoyable. Also, as wash mitts are often used by children between the ages of 24 and 48 months independently, there is a need for the solution coated onto the mitt to be extremely mild to the ocular mucosae to prevent tearing. The overall dimensions of the mitt need to be easy for toddlers to use, yet large enough to accommodate a larger adult hand. Also the mitt needs to have enough body wash to last throughout the bath. Because toddlers tend to take longer baths caregivers want a product that does not allow the soap to rinse out right away.

Unlike wet wipe compositions which are generally thought to be 70% or greater water by weight and typically 90% or greater, wash mitts typically have no more than 50% by weight. While personal care compositions comprising more than about 50% by weight of the composition of a liquid carrier, such as water, are within the scope of the present invention, it is preferred, with the embodiment of a mitt, the product contains no more than 50% water until the child first immerses the disposable cleaning implement or otherwise contacts it with water.

Ideal characteristics of the invention are that the solution is mild or tear-free, gentle for use on children, gentle on skin for less irritation, does not dry out skin, produces enough foam to make cleaning fun and be easy for children to use on themselves (easy to lather, rinse).

To ensure the mitt fit a wide range of hand sizes, the nonwoven fibrous sheet materials forming the mitt may contain elastomeric polymers. The elastomeric polymer may impart additional stretch characteristics to the polymeric network. Suitable elastomeric random block polymers include, for example, styrene-butylene-styrene (SBS) polymers, styrene-ethylene/butylene-styrene (SEBS) polymers, styrene-ethylene/propylene-styrene (SEPS), and combinations thereof, all commercially available as Kraton Polymers, Houston, Tex. Additional elastomeric random block polymers for use in the cleansing product can include semi-crystalline polyolefin polymers, such as commercially available from ExxonMobil, Houston, Tex., under the trade name Vistamaxx®. Typically, when used in the cleansing product, the elastomeric random block polymers are present in the product in an amount of from about 5% (by total weight product) to about 15% (by total weight product). If desired, the nonwoven web may also be imparted with texture on one or more of its surfaces. For instance, techniques for forming dual-textured spunbonded or meltblown materials are described in U.S. Pat. No. 4,659,609 issued to Lamers, et al. and U.S. Pat. No. 4,833,003 issued to Win, et al., which are both incorporated herein by reference to the extent they are consistent herewith.

The substrates, as disclosed herein, are illustrated by the following examples, which are not to be construed in any way as imposing limitations upon the scope thereof. On the contrary, it is to be clearly understood that various other embodiments, modifications, and equivalents thereof which, after reading the description herein, may suggest themselves to those skilled in the art without departing from the spirit and/or the scope of the appended claims.

### Test Methods

### Coefficient of Friction

Coefficient of Friction is obtained using the Kawabata Evaluation System (KES) test instrument KES model FB-4S. The KES instrument is available from Kato Tech Co., Ltd., Kyoto, Japan.

The sample is placed on a specimen tray, and a holding frame is placed over the specimen. The machine direction measurement is taken first. Two probes, one to measure the coefficient of friction (reported as MIU) and one to measure the surface roughness (reported as SMD) are placed on the sample. The probe for measurement of surface roughness is made of a steel wire with a diameter of 0.5 mm. The coefficient of friction is measured using a probe with 10 pieces of steel wires each 0.5 mm in diameter, and is designed to simulate the human finger. The sample is moved forward and backward underneath the two probes at a constant rate of 0.1 cm/sec. The measurement is taken for 2 cm over the surface. The distance or displacement of the probe is detected by a potentiometer. The coefficient of friction probe is detected by a force transducer. The vertical movements of the surface roughness probe are detected by a transducer. The displacement (distance) of the sample (L, cm) vs. the coefficient of friction (MIU-unitless) and surface roughness (SMD-Pm) are plotted. The sample is then rotated 90 degrees and tested again to provide the cross machine direction measurements. The following settings were used:

Friction sensitivity=2×5

Roughness Sensitivity=2×5

Static Load=25 g

To determine the coefficient of friction the test instrument KES model FB-4S uses the following analysis, wherein MIU is the mean value of the coefficient of friction (dimensionless) and MMD is the mean deviation of MIU (dimensionless).

The values are defined by

MIU( μ̅)=1X∫₀^{x}μdx

MMD=1/X∫₀^{x}|μ− μ̅|dx

where
μ=friction force divided by compression force
μ̅=mean value of μ
x=displacement of the probe on the surface of specimen, cm
x=maximum travel used in the calculation, 2 cm
   The physical meanings of the surface test parameters are as follows.

### Coefficient of Friction (MIU)

Higher values of MIU indicate more drag on the sample surface.

### Mean Deviation of MIU (MMD)

Higher values indicate more variation or less uniform on the sample surface.

### Examples

### Example 1

### Disposable Wet Wipe Formulation

Fibrous nonwoven structures containing wood pulp fibers and meltblown polypropylene fibers were produced in accordance with the process described using the process as described in, for example, U.S. Pat. No. 4,100,324 issued to Anderson et al.; U.S. Pat. No. 5,508,102 issued to Georger et al.; and in U.S. Patent Application Publication US 2003/0211802 by Keck et al., all of which are herein incorporated by reference. In the process, secondary pulp fibers, CF405 pulp commercially available from the Weyerhauser Company, are suspended in an air stream and contacted with two air streams of meltblown fibrous materials, Metocene MF650X, commercially available from Basell USA Inc., Pasadena, Tex., impinging the air stream containing secondary pulp fibers. The merged streams were directed onto a forming wire and collected in the form of a fibrous nonwoven coform strucuture.

The 60 grams per square meter basesheet was machine-converted into sections of continuous web 5.5″ wide by 56″ long with perforations every 7″ which were adhesively joined, fan-folded and applied with the cleansing composition at 330% add-on to yield a fan-folded stack of wet wipes. A liquid cleansing composition containing 0.64% Planatpon LGC Sorb, a surfactant blend containing sodium lauryl glucose carboxylate and lauryl glucoside available from COGNIS Corporation, 0.45% Purox S (sodium benzoate available from Ashland Chemical), 0.1% Neolone 950 (methylisothiazolone from Rohm & Haas), malic acid and at least one additive is prepared. Comparative Formulations 1-6 do not contain the sarcosine based surfactant of the present disclosure while Exemplary Formulations 7-8 contain sodium lauroyl sarcosinate, an exemplary sarcosine based surfactant. The cleansing compositions used to prepare the wet wipes with different treatments had added thereto either a sarcosine based surfactant of the present disclosure, no additional additive, or alternative additives evaluated with improved glide. The various formulations are described below in Table 1.

TABLE 1
Exemplary and Comparative Formulations

| | | Form. | Form. | Form. | Form. | Form. | Form. | Form. | Form. |
|---|---|---|---|---|---|---|---|---|---|
| Trade | | 1 (wt. | 2 (wt. | 3 (wt. | 4 (wt. | 5 (wt. | 6 (wt. | 7 (wt. | 8 (wt. |
| Name | INCI Name | %) | %) | %) | %) | %) | %) | %) | %) |
| Water | Water | 98.41 | 98.71 | 98.85 | 98.75 | 98.73 | 95.75 | 98.68 | 98.21 |
| Plantapon | Sodium Lauryl | 0.64 | 0.64 | — | 0.30 | — | — | 0.47 | 0.64 |
| LGC Sorb | Glucose | | | | | | | | |
| | Carboxylate and | | | | | | | | |
| | Lauryl Glucoside | | | | | | | | |
| Corum | Sodium Lauroyl | — | — | — | — | — | — | 0.20 | 0.50 |
| 6130 | Sarcosinate | | | | | | | | |
| Emulgin | Polysorbate 20 | 0.20 | — | 0.30 | 0.30 | — | — | — | — |
| SML 20 | | | | | | | | | |
| | Surfactant blend¹ | — | — | — | — | — | 4.152 | — | — |
| Merquat | Polyquaternium-7 | — | — | 0.20 | — | 0.30 | — | — | — |
| 740 | | | | | | | | | |
| Betafin | Betaine | — | — | — | — | 0.50 | — | — | — |
| BP20 | | | | | | | | | |
| Purox S | Sodium Benzoate | 0.45 | 0.45 | 0.45 | 0.45 | 0.45 | — | 0.45 | 0.45 |
| Neolone | Methylisothiazolione | 0.10 | 0.10 | 0.10 | 0.10 | 0.10 | — | 0.10 | 0.10 |
| 950 | | | | | | | | | |
| Malic | Malic Acid (pH | 0.10 | 0.10 | 0.10 | 0.10 | 0.10 | 0.10 | 0.10 | 0.10 |
| Acid | ~4.5) | | | | | | | | |

¹Wetting composition used on HUGGIES ® wet wipes (commercially available from Kimberly-Clark Corporation).

Consumers have indicated that the wipe containing comparative formulation 5 having no additional additive did not glide across the skin very well and bunched and rolled across the baby's skin during use. Exemplary formulations 7 and 8 including the sodium lauroyl sarcosinate have better glide across the skin. To quantify the perceived improvement in glide, the formulations were submitted for coefficient of friction testing as described herein.

TABLE 2
Coefficient of Friction Values

| Formulation | Coefficient of Friction |
|---|---|
| 1 | 0.4186 |
| 2 | 0.4086 |
| 3 | 0.3932 |
| 4 | 0.3886 |
| 5 | 0.3806 |
| 6 | 0.3804 |
| 7 | 0.3696 |
| 8 | 0.3370 |

As shown in Table 2, coform cleansing substrates containing 0.5% of Corum 6130 having sodium lauroyl sarcosinate had a significantly lower coefficient of friction than the comparative formulations that were prepared. The formulations including the sarcosine based surfactant all had coefficient of friction of less than 0.38. The comparative formulations with either no additional additive or alternative additives all had a coefficient of friction of greater than 0.38. Lower static coefficient of friction values indicate lower shear forces between the skin and the material, an increased feeling of gentleness of the wipe and improved ability of the wipe to glide across the skin.

The findings of these studies are significant and unexpected. The use of sarcosinates in the prior art has been described as anionic lathering surfactants in addition to sulfates, isethionates, taurates, phosphates, lactylates, glutamates, and mixtures thereof. The prior use relies on traditional skin conditioning agents such fatty acids, esters of fatty acids, fatty alcohols, ethoxylated alcohols, polyol polyesters, glycerin mono-esters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, synthetic waxes, polyolefinic glycols, polyolefinic monoester, polyolefinic polyesters, cholesterols, cholesterol esters and mixtures thereof to impart to desired skin feel.

### Example 2

### Disposable Wash Mitt

In this example, wash mitts having a cleansing formulation with the sarcosine based surfactant described herein were prepared. Within this example a wash mitt of the following shape and dimensions was prepared having a length of 165 mm, a width at widest point of 127 mm, a width at necking of 101 mm, perimeter stitch/25 mm and lock stitch of 6 stitches/25 mm, distance of perimeter stitch from edge 4 mm and body wash weight of 7.2 grams. The basesheet is a 110 grams per square meter, three-layer, nonwoven laminate as described in U.S. Pat. No. 6,946,413 issued to Lange et al, incorporated herein by reference to the extent they are consistent herewith. The outer layers give the basesheet a soft, cloth-like feel while the center layer of polymer filaments and meltblown material gives elastomeric properties.

Once converted, the dry mitts can be coated with the exemplary formulations listed in Table 3 below to the target weight of 7.2 grams.

TABLE 3
Solutions for Wash Mitt Products Containing Sodium Lauroyl Sarcosinate

| | | Formulation | Formulation | Formulation |
|---|---|---|---|---|
| | | 11 | 12 | 13 |
| Trade Name | INCI Name | (wt. %) | (wt. %) | (wt. %) |
| Water | Water | 48.090 | 45.150 | 30.650 |
| Plantaren 2000 N | Decyl Glucoside | 19.000 | 19.000 | 19.000 |
| UP | | | | |
| Velvetex BA 35 | Cocamidopropyl | 6.000 | 6.000 | 6.000 |
| | Betaine | | | |
| Cetiol HE | PEG-7 Glyceryl | 2.000 | 2.000 | 2.000 |
| | Cocoate | | | |
| Glycerin | Glycerin | 1.000 | 1.000 | 1.000 |
| Crodasinic LS- | Sodium Lauroyl | 2.500 | 5.000 | 15.000 |
| 95 | Sarcosinate | | | |
| Plantapon ACG | Sodium Cocoyl | 15.000 | 15.000 | 15.000 |
| 50 | Glutamate | | | |
| | Coco Fatty Acids | | | |
| | Isopropyl Alcohol | | | |
| | Water | | | |
| Elestab FL-15 | Butylene Glycol | 2.000 | 2.000 | 2.000 |
| | Glycerin | | | |
| | Chlorphensin | | | |
| | Methylparaben | | | |
| | Fragrance | 1.200 | 1.200 | 1.200 |
| dl-Panthenol | Panthenol | 1.000 | 0.100 | 0.100 |
| Citric Acid | Citric Acid | 0.610 | 0.450 | 0.450 |
| Vitamin E | Tocopherol Acetate | 0.100 | 0.100 | 0.100 |
| Acetate | | | | |
| Versene NA2 | Disodium EDTA | 0.100 | 0.100 | 0.100 |
| Sodium | Sodium Metabisulfite | 0.400 | 0.400 | 0.400 |
| Metabisulfite | | | | |
| Sodium Chloride | Sodium Chloride | 1.000 | 1.000 | 1.000 |

Other modifications and variations to the appended claims may be practiced by those of ordinary skill in the art, without departing from the spirit and scope as set forth in the appended claims. It is understood that aspects of the various embodiments may be interchanged in whole or in part. The preceding description, given by way of example in order to enable one of ordinary skill in the art to practice the claimed invention, is not to be construed as limiting the scope of the invention, which is defined by the claims and all equivalents thereto.

What is claimed is:

1. A substrate for imparting a perceivable aesthetic feel to skin comprising:
a basesheet material; and
a cleaning composition including a sarcosine based surfactant.

2. The substrate of claim 1 wherein the sarcosine based surfactant is a sarcosinate surfactant is selected from ammonium cocoyl sarcosinate, ammonium lauroyl sarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium palmitoyl sarcosinate, sodium sarcosinate, TEA-cocoyl sarcosinate, TEA-lauroyl sarcosinate, TEA-oleoyl sarcosinate, TEA-palm kernel sarcosinate, and combinations thereof.

3. The substrate of claim 1 wherein the sarcosine based surfactant is sodium lauroyl sarcosinate.

4. The substrate of claim 1 wherein the cleansing composition comprises from about 0.05% (by weight of the cleansing composition) to about 10% (by weight of the cleansing composition) of the sarcosine based surfactant.

5. The substrate of claim 4 wherein the cleansing composition comprises from about 0.5% (by weight of the composition) to about 5% (by weight of the composition) of the sarcosine based surfactant.

6. The substrate of claim 1 wherein the substrate is selected from wipes, mitts, absorbent articles, bath tissues, or cloths.

7. The substrate of claim 4 wherein the substrate is a wet wipe.

8. The substrate of claim 7 wherein the cleansing composition further comprises from about 75% (by weight of the composition) to about 99.5% (by weight of the composition) of water.

9. The substrate of claim 1 wherein the cleaning composition further comprises an additional surfactant selected from surfactants including nonionic, anionic, cationic, amphoteric, zwitterionic, or combinations thereof.

10. The substrate of claim 9 wherein the additional surfactant is alkyl polyglucoside.

11. The substrate of claim 9 wherein the additional surfactant is sodium lauryl glucose carboxylate.

12. The substrate of claim 7 wherein the cleansing composition comprises from about 0.01% (by weight of the cleansing composition) to about 10% (by weight of the cleansing composition) of the sarcosine based surfactant.

13. The substrate of claim 1 having a static coefficient of friction value of less than about 0.40.

14. The substrate of claim 1 having a static coefficient of friction value of between about 0.30 to about 0.37.

15. The substrate of claim 1 wherein the wet wipe provides less friction and less irritation to the skin due to less skin glide.

16. The substrate of claim 1 wherein the cleansing composition further comprises a skin benefit agent selected from the group consisting of a quaternary ammonium material, a particulate, a rheology modifier, a moisturizer, a film former, a slip modifier, a surface modifier, a skin protectant, a sunscreen, and combinations thereof.
